(19) United States
(12) Patent Application Publication
(10) Pub. No.: US 2002/0025348 A1
(43) Pub. Date: Feb. 28, 2002

(54) Treatment for inflammatory bowel disease (IBD) and related conditions

(76) Inventors: Amaresh Basu, San Diego, CA (US); Keith Hoffman, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/875,299

(22) Filed: Jun. 5, 2001

Related U.S. Application Data
(60) Provisional application No. 60/173,006, filed on Dec. 23, 1999.

(51) Int. Cl. A61K 35/78

(52) U.S. Cl. 424/735; 424/736; 424/757; 424/756; 424/769

(57) ABSTRACT

Compositions, including formulations, of plants for the treatment of Inflammatory Bowel Disease, Irritable Bowel Syndrome, and inflammatory and other related conditions, such as arthritis, are disclosed. The formulations consist of one or both of the two herbs Chen Pi (Citrus reticulata) and Wu Mei (Prunus mume), which, according to the invention, possess significant cytokine TNF-α inhibitory activity. The formulations also may include one or more of the following herbs which, according to the invention, inhibit PGE₂: Hou Po (Magnoliae officinalis), Huang Bai (Phellodendron chinense), Huang Lian (Coptis chinensis), Huo Xiang (Agastaches rugosa), Pao Jiang (Zingiberis officinalis), Qin Pi (Fraxinus rynchophylla), and Zhi Gan Cao (Glycyrhizae inflata).

### PRIORITY CLAIM

The present application is a National Phase entry of PCT Application PCT/US00/34792, entitled “TREATMENT FOR INFLAMMATORY BOWEL DISEASE (IBD) AND RELATED CONDITIONS,” and filed on Dec. 20, 2000, which claims priority from U.S. Provisional Application Serial No. 60/173,006, also entitled “TREATMENT FOR INFLAMMATORY BOWEL DISEASE (IBD) AND RELATED CONDITIONS,” and filed on Dec. 23, 1999.

### BACKGROUND OF THE INVENTION

1. Field of the Invention

The present invention relates to novel formulations of plants and extracts thereof to be used for the treatment of bowel disorders. More specifically, the formulations of the invention can be used to treat Inflammatory Bowel Disease (IBD), and related conditions such as Irritable Bowel Syndrome (IBS), and other inflammatory disorders such as arthritis. The plants have been selected for their specific inhibition of the inflammatory mediators implicated in the pathogenesis of IBD, arthritis, and related conditions.

2. Background of the Invention

Inflammatory Bowel Disease is a heterogeneous group of diseases that have a common manifestation of (gut) mucosal inflammation. In general, IBD encompasses two major forms of intestinal inflammation: ulcerative colitis and Crohn's disease, known also as Crohn's ileitis, regional enteritis, or granulomatous colitis. Estimates place the domestic prevalence of these conditions between one and two million patients, with similar rates in other northern European countries [Crohn's & Colitis Foundation of America 1/99 Update]. The clinical and histopathologic features of IBD are well characterized; however the etiology and pathogenesis of IBD are still subjects of intense research. Currently, a variety of medical treatment modalities are used, with moderate success, to both control active “flare-ups” of IBD as well as to maintain remission(s). Aminosalicylate preparations such as sulfasalazine and mesalamine are the most common anti-inflammatory agents which are used to control ulcerative colitis and, to a lesser extent, Crohn's disease. While the specific mechanism remains undefined, inhibition of eicosanoid mediators such as prostaglandins and thromboxanes is the probable mechanism of action [Stein R B, Hanaur S B: Medical Therapy for Inflammatory Bowel Disease. GI Clin N Amer 1999;28(2):297-321]. Other typical treatments include corticosteroids and antibiotics such as metronidazole and ciprofloxacin for acute flares of disease. The other large category of drugs used in IBD is the immunomodulators, including azathioprine, methotrexate, and cyclosporine, the efficacy of which are principally related to their ability to inhibit T-cell related immune response and inflammatory cytokine cascades [Stotland B R, Lichtenstein M D: Newer Treatments for Inflammatory Bowel Disease. Primary Care 1996;23(3):577-608]. These treatments, unfortunately, induce worrisome side effects.

As mentioned, the causes of IBD remain obscure. A currently accepted hypothesis is that IBD represents an interaction between genetic and environmental factors, and implicates T-cell dysregulation, specifically an abnormally severe T-cell inflammatory response to mucosal antigens. (Commensal gut flora is implicated as the source of such antigens.) Regardless of the initial insult that activates the immune system, the inflammatory cascade which follows has been characterized. The Tₕ cell cytokines IL-1 and TNF-α are considered to be central to the pathogenesis of IBD [Papadakis K A, Targan S R: Current theories on the causes of inflammatory bowel disease. Gastroent Clin N Am 1999;28(2):283-96]. Further down the inflammatory cascade are the eicosanoid products of arachidonic acid, particularly Prostaglandin E2 (PGE₂) and Leukotriene B4 (LTB₄), which have been found in high levels in IBD patients [Stotland B R, Lichtenstein M D: Newer Treatments for Inflammatory Bowel Disease. Primary Care 1996;23(3):577-608].

The relatively recent discovery and characterization of cytokines and understanding of their part in the inflammatory cascade has prompted research on the role of cytokines in the pathogenesis of inflammatory diseases. Proinflammatory cytokines, such as tumor necrosis factor (TNF)-alpha and interleukin-1 (IL-1) play a major part in stimulating the inflammatory response. Overactivity of TNF has been associated with synoviocyte proliferation, neo-angiogenesis, the recruitment of inflammatory cells, and the production of degradative enzymes. These findings have stimulated the development of anticytokine therapies. Further investigation has shown that the signs of inflammatory disease can be abrogated when certain proinflammatory cytokines, such as TNF and IL-1, are neutralized by monoclonal antibodies, naturally occurring cytokine antagonists, or cytokine receptor blockers. For example, studies have shown that TNF-alpha antagonists afford beneficial treatment effects in patients with rheumatoid arthritis (RA) and Crohn's Disease (a form of IBD), and these results have been confirmed in larger clinical trials. In fact, the TNF-alpha monoclonal antibody infliximab (Remicade, Centocor, Malvern, Pa.), has been approved for use in Crohn's Disease and in combination with methotrexate (MTX) for the treatment of RA. Etanercept (Enbrel, Wyeth Ayerst, Philadelphia, Pa.), a recombinant protein that binds TNF-alpha is approved for RA, both as monotherapy and in combination with MTX.

In short, while the initial precipitating factors of IBD are not well known, the mediators responsible for its persistence and exacerbations are better understood. It is also clear that current therapy is far from perfected, and there is a significant need for newer remedies with reduced side-effect profiles.

Irritable Bowel Syndrome, unlike IBD, does not have any characteristic histopathological changes (or at least none that has been detected with current medical technology), but rather is a functional disorder of disturbed gut motility and/or abdominal pain perception possibly linked to cytokines and/or other inflammatory cascades [Collins S M, Barbara G, Vallance B, Stress, inflammation and the irritable bowel syndrome. Can J Gastroenterol 1999: 13; A:47A-49A; Bueno L, Fioramonti J, Effects of inflammatory mediators on gut sensitivity. Can J Gastroenterol 1999:13; A:42A-46A]. IBS also tends to occur in IBD patients who are in remission from their IBD symptomologies [Collins S M, et al. Putative inflammatory and immunological mechanisms in functional bowel disorders. Baillieres Best Pract Res Clin Gastroenterol 1999: 13; 429-436]. IBS has a reported prevalence generally between 15-25% in both the industrialized and developing world [Malcolm A, Kellow J E. Irritable bowel syndrome. MJA 1998;169:274-279]. The diagnosis of IBS is made by history alone, and is generally characterized by painful defecation and altered stool frequency/consistency. Therapy has generally consisted of dietary modification, anti-diarrhea preparations, or antispasmodic preparations. Newer treatments include opioid κ receptor antagonists and serotonin 5HT₃ and 5HT₄ receptor antagonists with the hope that this will reduce the heightened perception of visceral pain which may contribute to IBS [Farthing M: New drugs in the management of irritable bowel syndrome. Drugs 1998;56(1):11-21]. Ultimately, however, no single drug has proven to be effective in the treatment of the IBS symptom complex [Scarpignato C, Pelosini I: Management of irritable bowel syndrome: novel approaches to the pharmacology of gut motility. Can J Gastroenterol 1999;13suppl:50A-65A]. Glaxo Wellcome Inc.'s Lotronex (Alosetron), a 5HT3 antagonist, was the first FDA-approved drug for treatment of IBS. Recently, however, the drug was pulled from the market due to life-threatening cases of ischemic colitis associated with taking the drug. Much the same as IBD, the pathophysiology of IBS is not well understood and there are a variety of tentative hypotheses to explain the phenomenon. Most researchers agree that there is a heightened sensitivity to visceral pain perception in IBS. The concept of “peripheral sensitization” involves a reduction in the threshold and an increase in the gain of the transduction processes of primary afferent neurons. This may be mediated by any or a combination of a variety of substances, including monoamines (both catecholamines and indoleamines), substance P, and variety of cytokines and prostanoids including the E-type prostaglandins [Mayer E A, Gebhart G F: Basic and clinical aspects of visceral hyperalgesia. Gastroenterology 1994;107:271-293]. While this and other explanations are being investigated, the fact remains that current treatments are inadequate and riddled with problematic side effects.

Treatment options that may address one or more of the multiple therapeutic targets associated with the bowel-related disorders described above, are therefore desirable.

Particularly, there is an ongoing need to develop agents which can ameliorate the pathological biochemistries and symptomologies characteristic of IBD, IBS, arthritis, and other related conditions. More particularly, there exists a need for an effective, alternative treatment and related treatment regime options for individuals who are afflicted with IBD, IBS, and/or related disorders. The instant invention meets these and other needs.

### SUMMARY OF THE INVENTION

The present invention includes compositions and formulations of plants for the treatment of Inflammatory Bowel Disease, Irritable Bowel Syndrome, and related conditions. The formulations consist of one or both of the two herbs Chen Pi (Citrus reticulata) and Wu Mei (Prunus mume), which, according to the invention, possess significant cytokine TNF-α inhibitory activity. The formulations also may include one or more of the following herbs which, according to the invention, inhibit TNF-α induced PGE₂: Hou Po (Magnoliae officinalis), Huang Bai, which is also often referred to as Huang Bo (Phellodendron chinense), Huang Lian (Coptis chinensis), Huo Xiang (Agastaches rugosa), Pao Jiang (Zingiberis officinalis), Qin Pi (Fraxinus rynchophylla), and Zhi Gan Cao (Glycyrhizae inflata).

In developing an optimal remedy for bowel related disorders, several existing herbal preparations were examined, including Tong Xie Yao Fang, a preparation of six herbs traditionally used in Chinese medicine for the treatment of IBS-like symptoms [Zhang, J, “A complete Works of Jingyue”—see page 540 of “Chinese English manual of common-used prescriptions in Traditional Chinese Medicine”, Canton Science and Technology Press, China]. Also examined was a twenty-herb formulation [Bensoussan A. et al. Treatment of irritable bowel syndrome with Chinese herbal medicine: a randomized controlled trial. JAMA. Nov. 11, 1998;280(18):1585-9], comprising Chai HU, CHen Pi, CHen Qian Zi, Bai Shao, Bai Zhu, Bai Zhi, Dang Shen, Fang Feng, Fu LIng, Hou Po, Huang Bai, Huang Lian, Huo Xiang, Mu Xiang, Pao Jiang, Qin Pi, Yin Chen, Yi Yi Ren, Zhi Gan Cao, and Wu Wei Zhi.

The present pharmacological investigation of the specific herbal components of these mixtures yielded unexpected results. Extracts of these formulations were examined for both inhibition of TNF-α secretion and inhibition of PGE₂ release (FIGS. 1 and 2). Based on the preliminary results, the activities of the individual herbal components were examined. Two of the herbs from the six-herb Tong Xie Yao Fang formulation, Chen Pi (Citrus reticulata) and Wu Mei (Prunus mume), showed significant inhibition of LPS-induced TNF-α secretion as assayed in human peripheral blood mononuclear leukocyte cells (FIG. 3). Seven of the twenty herbs from the distinct twenty-herb formulation exhibited significant inhibition of TNF-α induced release of PGE₂ as assayed in human HeLa S3 epitheloid cervix carcinoma cells: Hou Po (Magnoliae officinalis), Huang Bai (Phillodendron chinense), Huang Lian (Coptis chinensis), Huo Xiang (Agastaches rugosa), Pao Jiang (Zingiberis officinalis), Qin Pi (Fraxinus rynchophylla), and Zhi Gan Cao (Glycyrhizae inflata). Given the role of these inflammatory mediators in the pathogenesis of IBD and related disorders (and the safe history of use of such plants), the invention describes a formulation using either or both of the two herbs exhibiting cytokine TNF-α inhibitory activity, either alone or in combination with one or more of the seven herbs exhibiting inhibition of PGE₂ release.

Additionally, as mentioned, there is not yet a satisfactory explanation regarding the pathophysiology of IBS. The pharmacological activities of the herbs listed above, however, suggests that they may be particularly useful in treatment of IBS and/or IBD. Acute enteric inflammation, which occurs commonly in patients in remission from inflammatory bowel disease (IBD), is now considered a possible basis for symptom generation in IBS (Collins S M, Vallance B, Barbara G, Borgaonkar M: Putative inflammatory and immunological mechanisms in functional bowel disorders; Baillieres Best Pract Res Clin Gastroenterol; Oct. 13, 1999(3):429-36). Thus, reduction in inflammatory agents like TNF-α and prostaglandins by the herbs used here would have significant effect in the treatment of inflammatory diseases like IBS and IBD. Their unexpected and significant inhibition of cytokine TNF-α and PGE₂ leads to the conclusion that among the many other herbs used for IBS, the combination of the nine herbs identified according to the invention (in total or in part) may be particularly efficacious for the treatment of IBD and IBS. This efficacy may be due, in part, to the prevention of the visceral hypersensitization and hyperalgesia which is potentiated by both cytokines and E-type prostaglandins.

### BRIEF DESCRIPTION OF THE DRAWINGS

The accompanying drawings, which are incorporated in and form part of the specification, merely illustrate embodiments of the present invention. Together with the remainder of the specification, they are meant to serve to explain the principles of the invention to those of skill in the art. In the drawings:

FIG. 1 depicts a graphic representation of the effect of various plant extracts on LPS-induced TNF-α release.

FIG. 2 depicts a graphic representation of the effect of various plant extracts on TNF-α induced PGE₂ release.

FIG. 3 depicts a graphic representation of the effect of various plant extracts on Inhibition of LPS-induced TNF-α.

FIG. 4 depicts a graphic representation of the effect of various plant extracts on inhibition of TNF-α-induced PGE₂ release (Yi Yi Ren being mislabeled as Yi Yl Ren).

### DETAILED DESCRIPTION OF THE PREFERRED EMBODIMENTS

The present inventors have determined that certain plant extracts can significantly inhibit TNF-α induced prostaglandin E₂ (PGE₂) release and/or LPS-induced TNF-α release. The specific plant extracts may be used alone, or in combination, or with other ingredients, for the treatment of a variety of bowel disorders. One object of the present invention is to provide novel formulations of plants for the treatment of bowel disorders. Another object of the invention is to provide novel formulations of plants for the treatment of IBD and IBS, and related disorders.

Various solvents and/or solvent systems may be used in making the present extracts and of the present formulations. For example, water may be used, as may be hydroalcoholic solvent systems (for example, mixtures of water and ethanol, water and methanol, or water and any water-miscible alcohol), hexane(s), CH₂Cl₂, and combination thereof, and like polar, apolar, and/or aprotic solvents and solvent systems. Additionally, the following solvents may be used alone or as part of a solvent system: hydroxylic solvent (e.g., water, C₁-C₈ alcohols, preferably methanol, ethanol, isopropanol), a polar aprotic solvents (e.g., acetonitrile, dimethylformamide (DMF), dimethyl sulfoxide, acetone), or relatively non-polar organic solvent (e.g., benzene, toluene, liquid alkanes, preferably C₄-C₈ alkanes), or any suitable combination thereof. Also, salts, detergents, and other additives may be added to these solvents and/or solvent systems to facilitate extraction, as will be appreciated by those of skill in the art.

The extract(s) produced according to the methods of the present invention advantageously may be administered to an individual in a dose containing a pharmaceutically-effective amount of the extract(s), or component(s) found therein. This administration can be through any effective route. It is contemplated that administration may be effected, for example, preferably orally, and may also be administered intramuscularly, subcutaneously, intraperitoneally, transdermally, transmucosally, buccally, or through inhalation or pulmonary infusion. Dosages that are contemplated for a 70 kg adult human range from a lower limit of 10, 25, 50, 100, 150, 200, or 250 mg to an upper limit of 750, 1000, 1500, 2000, 2500, 3000, 4000, 5000, or up to 10,000 mg. of the extracts used in Examples 1 and 2, or other extracts. Preferred dosages for a 70 kg human are from about 200, 500, or 750 mg to about 2000, 3000, 4000, or 5000 mg. These dosages can be administered once, twice or up to four times per day, or two or more dosages may be combined into a controlled release formulation or dermal or mucosal patch of known type or may be administered through an infusion device over a period of time. The dose may also be tailored to achieve a desired effect, but will depend on such factors as weight, diet, concurrent medication and other factors which those skilled in the medical arts will recognize.

The present invention also encompasses pharmaceutical compositions comprising a pharmaceutically acceptable carrier prepared for storage and subsequent administration, which have a pharmaceutically effective amount of the extract disclosed above in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

These compositions may be formulated and used as tablets, capsules or liquid elixirs for oral administration; suppositories for rectal administration; sterile solutions, suspensions for injectable administration; or other formulations known in the art. Suitable excipients are known in the art and include, for example, water, saline, dextrose, mannitol, lactose, lecithin, albumin, sodium glutamate, cysteine hydrochloride, and the like. In addition, if desired, the pharmaceutical compositions may contain relatively small amounts of nontoxic auxiliary substances, such as wetting agents, pH buffering agents, and the like, as are known to persons of skill in the art.

In practicing the compositions of the invention, the formulated dosage may be used alone or in combination with other therapeutic or diagnostic agents. These products can be utilized in vivo, ordinarily in a mammal, preferably in a human, or in vitro. In employing them in vivo, the products or compositions can be administered to the mammal in any of a variety of manners known to persons skilled in the art, including parenterally, intravenously, subcutaneously, intramuscularly, colonically, rectally, nasally or intraperitoneally, and may employ any of a variety of dosage forms.

As used herein, the term “disorder” refers to any disorder, disease, condition, syndrome or combination of manifestations or symptoms recognized or diagnosed as a disorder. If modified by a phrase such as bowel or inflammatory or modified by one or more or a set of manifestations or symptoms, that usage of “disorder” refers to any such disorder, disease, condition, syndrome or combination of such manifestations or symptoms recognized or diagnosed as a such disorder.

As used herein, the term “pharmaceutically effective amount” refers to an amount sufficient to alleviate, in any degree or manner, one or more of the manifestations or symptoms recognized or diagnosed as associated with the modifying disorder, the modifying manifestations, or the modifying symptom.

The various articles of the scientific and/or medical literature and texts cited herein are hereby incorporated by reference; each constitutes a part of the disclosure of this specification. Furthermore, while specific embodiments, and working and prophetic examples of the invention have been described in detail to illustrate the broad applicability and principles underlying the invention, it will be understood by those of skill in the art that the invention may be embodied otherwise without departing from such broad applicability and principles.

### EXAMPLE 1

As an example to illustrate, but not to limit the scope of, the invention extracts of specific species were produced and tested in in vitro assay systems indicative of certain cytokine and/or inflammatory cascades involved in bowel disorders. These extracts were obtained and the assays completed in the following manner.

Materials. Individual herbs were purchased from various traditional Chinese Medicine pharmacies in the People's Republic of China, Shanghai. While the quality of the herbs may vary slightly among the various commercial sources, the inventors have no preferred source from among traditional Chinese Medicine pharmacies in the People's Republic of China, Shanghai. The herbs were extracted and combined by methods familiar to those skilled in the art. The following five samples were prepared:

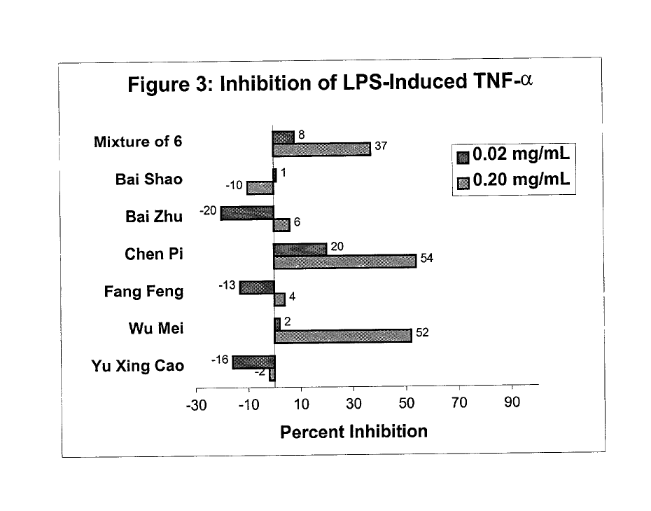

KL-015-05-F1: mixture of the six herbs (single water extract), as listed in FIG. 3, of the Tong Xie Yao Fang formulation: Bai Shao (Paeonia lactiflora), Bai Zhu (Atractylodes macrocephala), Chen Pi (Citrus reticulata), Fang Feng (Saposhnikovia divaricata), Wu Mei (Prunus mume), and Yu Xing Cao (Houttuynia cordata).

KL-015-09-F2: mixture of same 6 herbs used to prepare KL-015-05-F1 (each herb extracted with water individually and mixed in equal ratio on a weight basis).

KL-015-09-F3 through F8: individual herbs, as listed in FIG. 3, of the Tong Xie Yao Fang formulation: Bai Shao (-F3), Bai Zhu (-F4), Chen Pi (-F5), Fang Feng (-F6), Wu Mei (-F7), and Yu Xing Cao (-F8).

KL-015-032-F1: commercially available product, to be compared to mixture of twenty herbs in KL-015-033-F1.

KL-015-033-F1: mixture of twenty herbs (single water extract)

KL-015-033-F2: mixture of same twenty herbs used to prepare KL-015-033-F1 (simulated gastric fluid extract).

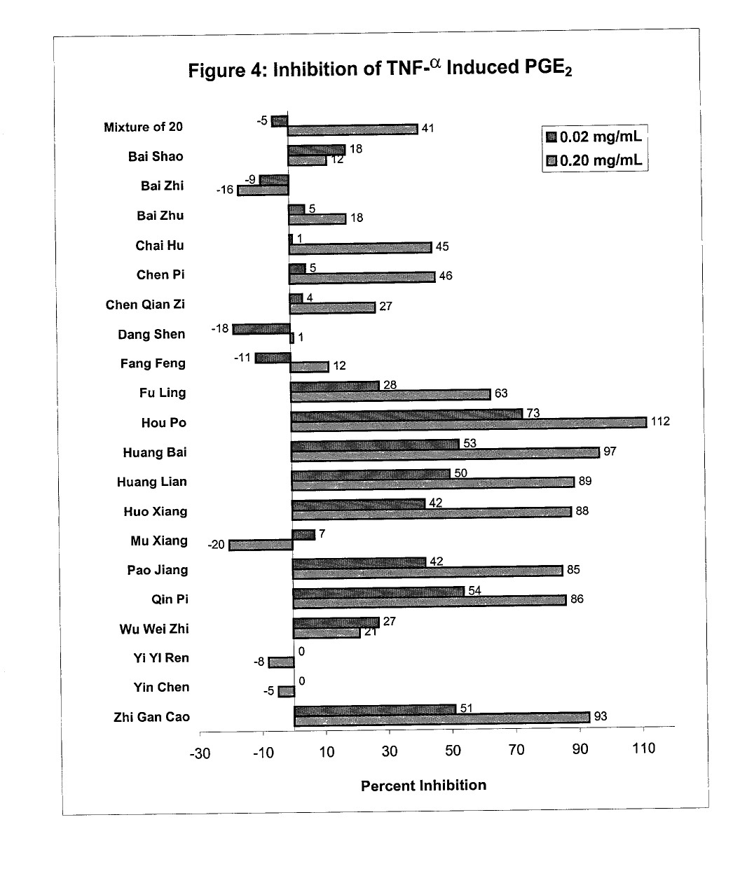

KL-015-033-F5 through F24: individual herbs, not in the order listed in FIG. 4, of the twenty-herb formulation of Bensoussan A. et al. Treatment of irritable bowel syndrome with Chinese herbal medicine: a randomized controlled trial. JAMA. Nov. 11, 1998;280(18):1585-9.

Test samples were prepared in 100% DMSO. Prior to analysis, the stock solutions were diluted in appropriate buffer for each assay. Final DMSO concentration was less than or equal to 1%.

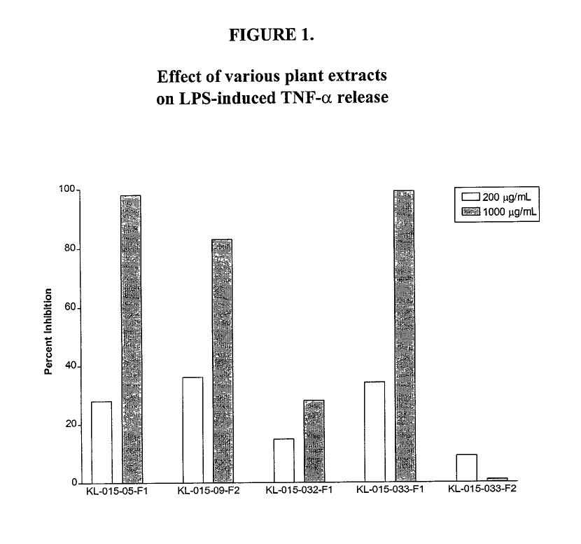

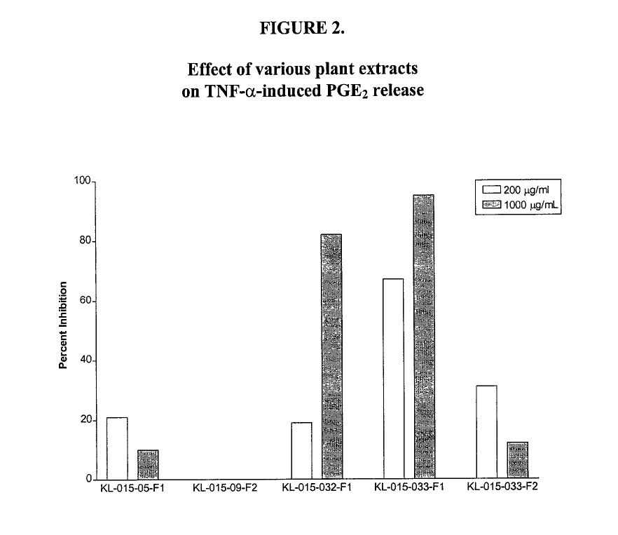

Assays. The inhibition of lipopolysaccharide (LPS) induced Tumor Necrosis Factoralpha (TNF-α) release in human peripheral blood mononuclear leukocyte (PBMCL) cells by various plant extracts is shown in FIG. 1. FIG. 2 provides a graphic representation of the inhibition of TNF-α induced prostaglandin E₂ (PGE₂) release in human epitheloid cervix carcinoma (HeLa S3) cells by the same plant extracts. Compounds that showed significant inhibition of LPS-induced TNF-α release were evaluated at the same concentrations for compound-induced cytotoxicity in PBMCL cells. No cytotoxicity was observed.

The results clearly indicate that only certain plants traditionally prescribed for bowel disorders possess anti-inflammatory activity. At a testing concentration of 1 mg/mL, both KL-015-05-F1 and KL-015-09-F2, a mixture of six herbs, inhibited LPS-induced TNF-α release, but not TNF-α-induced PGE₂ release. In contrast, KL-015-032-F1 inhibited TNF-α-induced PGE₂ release at 1 mg/mL, but was ineffective in the LPS-induced TNF-α assay (inhibiting release by only 28% at 1 mg/mL). The 20 herb mixture was effective in both assays when prepared as a water extract (KL-015-033-F1) and ineffective when prepared as a simulated gastric fluid extract (KL-015-033-F2).

### EXAMPLE 2

As a further example to illustrate, but not to limit the scope of, the invention, extracts of specific plant species were produced and tested in in vitro assay systems indicative of certain cytokine and/or inflammatory cascades thought to be involved in bowel disorders. To understand the role of each plant in the TNF-α-induced PGE₂ release assay and LPS-induced TNF-α assay, extracts of the individual plants were tested at a final concentrations of 0.02 and 0.2 mg/ml.

Materials. Individual herbs were purchased from commercial sources including various traditional Chinese Medicine pharmacies in the People's Republic of China, Shanghai. While the quality of the herbs may vary slightly among the various commercial sources, the inventors have no preferred source from among traditional Chinese Medicine pharmacies in the People's Republic of China, Shanghai. The herbs were extracted and combined by methods familiar to those skilled in the art. Samples were prepared as in, or in a manner comparable to that of, Example 1.

Assay. FIG. 3 shows the effect of individual plants, comprising KL-015-05-F1, on LPS-induced TNF-α release. The results clearly indicate that, among the individual plant extracts, only the extracts of KL-015-09-F5, Chen Pi, and KL-015-09-F7, Wu Mei, were effective in blocking LPS induced TNF-α levels (inhibiting TNF-α release by about 50% at a concentration of 0.2 mg/ml) while the extracts of other four plants were ineffective in blocking LPS induced TNF-α levels.

FIG. 4 shows the effect of individual plants, comprising KL-015-033-F1, on TNF-α-induced PGE₂ release. The results indicate that different extracts have varying degree of potency in blocking TNF-α induced PGE₂ levels. The most potent extracts were of those of Hou Po (Magnoliae officinalis), Huang Bai, which is also often referred to as Huang Bo (Phellodendron chinense), Huang Lian (Coptis chinensis), Huo Xiang (Agastaches rugosa), Pao Jiang (Zingiberis officinalis), Qin Pi (Fraxinus rynchophylla), and Zhi Gan Cao (Glycyrhizae inflata), each of which inhibited PGE2 release by 85% or more at a concentration of 0.2 mg/ml.

In summary, the results from the individual plants indicate that only some of the plants, comprising traditionally prescribed bowel formulations, have anti-inflammatory properties. The role and/or utility of other plants in the traditional formulas is beyond the scope of the present invention.

Two of the herbs from the Tong Xie Yao Fang formulation, Chen Pi (Citrus reticulata) and Wu Mei (Prunus mume), demonstrated significant inhibition of LPS-induced TNF-α secretion as assayed in human peripheral blood mononuclear leukocyte cells (FIG. 3). Furthermore, seven of the twenty herbs from the preparation used by Bensoussan et al.: Hou Po (Magnoliae officinalis), Huang Bai (Phellodendron chinense), Huang Lian (Coptis chinensis), Huo Xiang (Agastaches rugosa), Pao Jiang (Zingiberis officinalis), Qin Pi (Fraxinus rynchophylla), and Zhi Gan Cao (Glycyrhizae inflata), exhibited significant inhibition of TNF-α induced release of PGE₂ as assayed in human HeLa S3 epitheloid cervix carcinoma cells (FIG. 4). Given the role of these inflammatory mediators in the pathogenesis of IBD and related disorders (and the safe history of use), the invention describes a formulation using either or both of the two herbs exhibiting TNF-α inhibition in combination with one or more of the seven herbs exhibiting inhibition of PGE₂.

The inhibition of TNF-α production and/or inhibition of PGE₂ should be a viable means for treating a variety of other diseases related to inflammation, including arthritis. The present invention may also have therapeutic activity in the flowing areas due to said activities of the plants disclosed herein. These areas include, but are not limited to, 1) rheumatoid arthritis—both anti-inflammatory and antinociceptive, 2) obesity—particularly the insulin resistance that develops as a result of obesity and also the adipocyte metabolic dysregulation that predisposes people to obesity itself, 3) prevention of the progression of congestive heart failure (such as due to post-ischemia reperfusion injury), 4) hepatoprotective effects from alcoholic and viral injury, 5) periodontal disease and also periodontal disorders caused from smoking (the LPS-TNF-PGE2 axis is directly implicated in said disorders), 6) modulation of degenerative osteoarticular disease (joint aging), and, additionally, the plants disclosed herein may have significant anti-pyretic activities.

What is claimed is:

1. A composition of matter useful in the treatment of a disorder of the bowel, said composition comprising:
(a) a pharmaceutically effective amount of an extract of a plant selected from the plant Citrus reticulata, the plant Prunus mume, and any combination thereof; and
(b) a pharmaceutically effective amount of an extract of a plant selected from the plant Magnoliae officinalis, the plant Phellodendron chinense, the plant Coptis chinensis, the plant Agastaches rugosa, the plant Zingiberis officinalis, the plant Fraxinus rynchophylla, the plant Glycyrhizae inflata, and any combination thereof.

2. A composition of matter useful in the treatment of a disorder treatable via at least one of the following, the inhibition of TNF-α and the inhibition of PGE₂, said composition comprising:
(a) a pharmaceutically effective amount of an extract of a plant selected from the plant Citrus reticulata, the plant Prunus mume, and any combination thereof; and
(b) a pharmaceutically effective amount of an extract of a plant selected from the plant Magnoliae officinalis, the plant Phellodendron chinense, the plant Coptis chinensis, the plant Agastaches rugosa, the plant Zingiberis officinalis, the plant Fraxinus rynchophylla, the plant Glycyrhizae inflata, and any combination thereof.

3. The composition of claim 2, wherein the disorder is arthritis.

4. The composition of claim 2, wherein the disorder is selected from obesity, congestive heart failure, alcoholic injury, viral injury, periodontal disease, a periodontal disorder caused from smoking, degenerative osteoarticular disease, and fever.

5. The composition of matter of claim 1 or 2, wherein the composition comprises (a) a pharmaceutically effective amount of an extract of the plant Citrus reticulata; and (b) a pharmaceutically effective amount of an extract of a plant selected from the plant Magnoliae officinalis, the plant Phellodendron chinense, the plant Coptis chinensis, the plant Agastaches rugosa, the plant Zingiberis officinalis, the plant Fraxinus rynchophylla, the plant Glycyrhizae inflata, and any combination thereof.

6. The composition of matter of claim 1 or 2, wherein the composition comprises:
(a) a pharmaceutically effective amount of an extract of the plant Prunus mume; and
(b) a pharmaceutically effective amount of an extract of a plant selected from the plant Magnoliae officinalis, the plant Phellodendron chinense, the plant Coptis chinensis, the plant Agastaches rugosa, the plant Zingiberis officinalis, the plant Fraxinus rynchophylla, the plant Glycyrhizae inflata, and any combination thereof.

7. A method for treating a bowel disorder in a mammal, said method comprising
identifying the mammal as suffering from a bowel disorder;
administering the composition of matter of claim 1 or 2 to the mammal.

8. The method of claim 7, wherein the mammal is a human.

9. The method of claim 7, wherein the bowel disorder is Inflammatory Bowel Disease (IBD).

10. The method of claim 7, wherein the bowel disorder is Irritable Bowel Syndrome (IBS).

11. A method of treating a patient having a bowel disorder, comprising the step of orally administering to said patient a composition comprising (a) an extract of at least one of the following plants: Citrus reticulata, Prunus mume, and (b) an extract of at least one of the following plants: Magnoliae officinalis, Phellodendron chinense, Coptis chinensis, Agastaches rugosa, Zingiberis officinalis, Fraxinus rynchophylla, Glycyrhizae inflata.

12. A method of making a composition for treating bowel disorders comprising:
subjecting at least one anti-TNF-α herb to an extraction procedure, and
subjecting at least one anti-PGE₂ herb to an extraction procedure.

13. The method of claim 12, wherein the at least one anti-TNF-α herb is selected from the group consisting of Citrus reticulata and Prunus mume, and wherein the at least one anti-PGE₂ herb is selected from the group consisting of Magnoliae officinalis, Phellodendron chinense, Coptis chinensis, Agastaches rugosa, Zingiberis officinalis, Fraxinus rynchophylla, Glycyrhizae inflate.

14. A method of making a composition for treating arthritis, obesity, congestive heart failure, alcoholic injury, viral injury, periodontal disease, periodontal disorders caused from smoking, degenerative osteoarticular disease, and/or fever comprising:
subjecting at least one anti-TNF-α herb to an extraction procedure, and
subjecting at least one anti-PGE₂ herb to an extraction procedure.

15. The method of claim 14, wherein the at least one anti-TNF-α herb is selected from the group consisting of Citrus reticulata and Prunus mume, and wherein the at least one anti-PGE₂ herb is selected from the group consisting of Magnoliae officinalis, Phellodendron chinense, Coptis chinensis, Agastaches rugosa, Zingiberis officinalis, Fraxinus rynchophylla, Glycyrhizae inflata.

16. A method of making a composition for treating a disorder or disease comprising:
subjecting at least one anti-TNF-α herb to an extraction procedure, and
subjecting at least one anti-PGE₂ herb to an extraction procedure.

17. The method of claim 16, wherein the at least one anti-TNF-α herb is selected from the group consisting of Citrus reticulata and Prunus mume, and wherein the at least one anti-PGE₂ herb is selected from the group consisting of Magnoliae officinalis, Phellodendron chinense, Coptis chinensis, Agastaches rugosa, Zingiberis officinalis, Fraxinus rynchophylla, Glycyrhizae inflata.

18. The method of claim 16, wherein the disorder or disease is selected from the group consisting of arthritis, obesity, congestive heart failure, alcoholic injury, viral injury, periodontal disease, periodontal disorders caused from smoking, degenerative osteoarticular disease, and fever.
